(19) United States
(12) Patent Application Publication
(10) Pub. No.: US 2020/0315927 A1
(43) Pub. Date: Oct. 8, 2020

(54) SOLID COSMETIC COMPOSITION FOR EYE MAKE-UP INCLUDING FIBER

(71) Applicant: COSMAX, INC., Hwaseong (KR)

(72) Inventors: Su Bin KIM, Seongnam (KR); Eun Ju CHOI, Suwon (KR); Eun Ju SEO, Suwon (KR); Myeong Sam PAKR, Seoul (KR)

(21) Appl. No.: 16/837,621

(22) Filed: Apr. 1, 2020

(30) Foreign Application Priority Data
Apr. 5, 2019 (KR) 10-2019-0040360

(51) Int. Cl. A61K 8/02; A61K 8/96; A61K 8/25; A61Q 1/10

(52) U.S. Cl. CPC A61K 8/027; A61K 8/965; A61Q 1/10; A61K 8/0229; A61K 8/25

(57) ABSTRACT

The present invention relates to a stick formulation cosmetic composition for eye make-up comprising a fiber and a hectorite-based oil thickener, and a stick formulation cosmetic product manufactured using the composition. A cosmetic composition according to an aspect allows a fiber to attach to eyebrows, providing an excellent effect of making the eyebrows look full, and also has excellent sustainability and waterproof properties.

### TECHNICAL FIELD

The present invention relates to a solid cosmetic composition for eye make-up comprising a fiber.

### BACKGROUND ART

The eyebrows are the line of hair which grows in a bow shape above the eyes along the browridge. Each individual has their own unique eyebrows with different shapes, thickness and length, and the eyebrows together with the eyes are one factor that greatly determines the appearance of a person depending on their shape. For example, droopy eyebrows give a lethargic appearance, and eyebrows with the end lifted make the person appear angry, regardless of his intention. Thus, the eyebrows are modified by shaving or drawing to alter the shape.

Generally, compositions for eyebrow make-up should have the function of coloring, eyebrow covering, etc., spreadability allowing the compositions to be uniformly applied without powdering or smudging, and sustainability allowing the make-up not to be easily smudged by sweat and sebum but to long last.

Meanwhile, conventional compositions for eyebrow make-up come in pencil types, powder types, liquid types, etc., depending on the formulations thereof. Among these types, the pencil types are characterized by the convenience in use and waterproof properties, but have short-term sustainability because of smudging by skin sebum due to the characteristics of the formulation. The powder types exhibit a natural make-up effect, but have short-term sustainability because of powdering, etc. Finally, the liquid types remedy the drawback of the eyebrow make-up, which is short-term sustainability, by using conventional self-tanning type products to allow the make-up effect to last for several days even after cleansing, but are not uniformly applied during reapplication or lack coloring properties, etc.

### RELATED ART

### Patent Document

### Korean Patent Laid-open No. 2018-0036264

### SUMMARY OF THE INVENTION

### Technical Task

It is an object of the present invention to provide a stick formulation cosmetic composition for eye make-up comprising a fiber and a hectorite-based oil thickener.

However, the technical task to be achieved by the present invention is not limited to the aforementioned task, and other tasks that are not mentioned can be clearly understood by a person having ordinary skill in the art based on the following description.

### Means for Solving the Task

The present invention provides a stick formulation cosmetic composition for eye make-up comprising a fiber and a hectorite-based oil thickener.

According to an embodiment of the present invention, the fiber and the hectorite-based oil thickener are comprised in a weight ratio of 0.1:1 to 2.5:1.

According to another embodiment of the present invention, the composition further comprises a silicate-based film forming agent.

According to yet another embodiment of the present invention, the fiber is at least one selected from the group consisting of rayon, nylon, polyester, acrylic, polyurethane and polypropylene.

According to yet another embodiment of the present invention, the hectorite-based oil thickener is at least one selected from the group consisting of hectorite, disteardimonium hectorite, stearalkonium hectorite and quaternium-18 hectorite.

According to yet another embodiment of the present invention, the fiber is comprised in an amount of 1 to 10 wt %.

According to yet another embodiment of the present invention, the hectorite-based oil thickener is comprised in an amount of 0.1 to 5 wt %.

According to yet another embodiment of the present invention, the cosmetic composition has at least one formulation selected from the group consisting of a stick-type eyebrow product, a stick-type eyeliner and a stick-type eyeshadow.

According to yet another embodiment of the present invention, the silicate-based film forming agent is comprised in an amount of 1 to 30 wt %.

Also, the present invention provides a stick formulation cosmetic product for eyebrow using the cosmetic composition.

### Advantageous Effect

The cosmetic composition for eye make-up according to the present invention, which has a fiber dispersed in a stick formulation, has an effect of making eyebrows look full, allowing the fiber to apply to eyebrows upon eyebrow make-up using the composition. The cosmetic composition also includes a silicate-based film forming agent that forms a strong film membrane, reducing make-up removal by moisture and sebum after the application of the cosmetic composition.

### BRIEF DESCRIPTION OF THE DRAWINGS

FIG. 1 is a photograph showing the finished composition manufactured in Example 1;

FIG. 2 is a photograph showing the finished composition manufactured in Comparative Example 1;

FIG. 3 is a photograph showing the composition of Examples 1, 2 and 3 molded in a lower molding and an upper molding;

FIG. 4 is a photograph showing the composition of Comparative Examples 1, 2 and 3 molded in a lower molding and an upper molding;

FIG. 5 is a photograph showing the comparison result of application between the composition of Example 1 and a conventional formulation after application to eyebrows;

FIG. 6 is a graph showing the comparison result of sensory evaluation on volume between the composition of Example 1 and a conventional formulation after application to eyebrows;

FIG. 7 shows the result of color sustainability evaluation on an eyebrow product manufactured with the composition of the present invention; and

FIG. 8 shows the result of waterproof test on an eyebrow product manufactured with the composition of the present invention.

### DETAILED DESCRIPTION FOR CARRYING OUT THE INVENTION

Eyebrow products are a cosmetic product used for making eyebrows look full. Cosmetic compositions for eyebrow usually have the function of coloring, eyebrow covering, etc., spreadability allowing the composition to be uniformly applied without powdering or smudging, and sustainability allowing the composition not to be easily smudged by sweat and sebum but to long last. The cosmetic compositions are typically prepared in stick formulations.

In this regard, formulations such as browcara have been developed for the purpose of making eyebrows look fuller by applying cosmetics containing a fiber to the strands of eyebrows using an applicator. However, the formulations have powdering problems or low easy-cleaning properties. Further, since the cosmetics are provided in liquid phase, they have short expiration dates.

The inventors of the present invention have conducted studies to provide a cosmetic composition in a convenient and clean formulation, which does not need a separate applicator, while comprising a fiber in a cosmetic product for eyebrow as above, and confirmed that a stick formulation cosmetic product for eyebrow can be manufactured by combining a fiber and a hectorite-based oil thickener, thereby completing the present invention.

The present invention provides a stick formulation cosmetic composition for eye make-up comprising a fiber and a hectorite-based oil thickener.

The present invention provides a composition that can use a fiber that had not been used in stick formulation cosmetic products for eyebrow, and the fiber and the hectorite-based oil thickener may be comprised in a weight ratio of 0.1:1 to 2.5:1. When the fiber is used in an amount of less than 0.1 parts by weight with respect to 1 part by weight of the hectorite-based oil thickener, the composition has a slight effect of making eyebrows look full. When the fiber is used in an amount of more than 2.5 parts by weight, the composition has a high viscosity and thus may not be prepared in a stick formulation. More preferably, the fiber may be comprised in a weight ratio of 1:1 to 2:1.

The fiber may be at least one selected from the group consisting of rayon, nylon, polyester, acrylic, polyurethane and polypropylene, preferably rayon. The fiber, which is used as an adhesive and a bulking agent in the cosmetic composition, may be comprised in an amount of 1 to 10 wt % in the cosmetic composition. When the fiber is used in an amount of less than 1 wt %, the composition has a slight effect of making eyebrows look full. When the fiber is used in an amount of more than 10 wt %, the composition has a high viscosity and thus may not be suitable for being filled. Preferably, when the fiber is used in an amount of 1 to 5 wt %, the composition may have desirable hardness and viscosity for a stick formulation.

The hectorite-based oil thickener may be at least one selected from the group consisting of hectorite, disteardimonium hectorite, stearalkonium hectorite and quaternium-18 hectorite, preferably disteardimonium hectorite. The “hectorite-based oil thickener,” which helps the dispersion of an insoluble solid in a solution and serves as a viscosity modifier in the cosmetic composition, may be comprised in an amount of 0.1 to 5 wt % in the cosmetic composition. When the hectorite-based oil thickener is used in an amount of less than 0.1 wt %, the composition has a small amount of oil and thus may not be easily applicable to the skin. When the hectorite-based oil thickener is used in an amount of more than 5 wt %, the composition has low hardness and thus may have decreased skin spreadability. Preferably, when the hectorite-based oil thickener is used in an amount of 0.1 to 2 wt %, the composition may have desirable feeling of use and spreadability for a stick formulation.

According to an embodiment of the present invention, the composition further comprises a silicate-based film forming agent. The silicate-based film forming agent can form a strong film membrane when applied, thereby improving sustainability and ensuring waterproof properties after application.

The silicate-based film forming agent may be trimethylsiloxysilicate, phenylpropyldimethylsiloxysilicate, trimethylsiloxysilicate/dimethicone crosspolymer, dimethicone/vinyltrimethylsiloxysilicate crosspolymer or a mixture thereof, and may be comprised in an amount of 1 to 30 wt % with respect to the total weight of the cosmetic composition. When the content of the silicate-based film forming agent is out of the range, the cosmetic composition has hardness that is not suitable for being applied to the skin and thus may exhibit decreased spreadability.

The present invention may further comprise a silicone oil, a mixture, a synthetic pigment, a silicone wax and a mineral wax, in addition to the fiber, hectorite-based oil thickener and silicate-based film forming agent.

The silicone oil, which provides soft spreadability to the cosmetic composition or serves as a solvent, may be caprylylmethicone, cyclomethicone, cyclotetrasiloxane, cyclohexasiloxane, cycloheptasiloxane, decamethylcyclopentasiloxane, cyclotetrasiloxane, cyclotrisiloxane, dimethicone, capryldimethicone, caprylyltrimethicone, cetearylmethicone, hexadecylmethicone, hexylmethicone, laurylmethicone, myristylmethicone, phenylmethicone, stearylmethicone, stearyldimethicone, trifluoropropylmethicone, cetyldimethicone, polyphenylmethylsiloxane, dimethylpolysiloxane, methylphenylpolysiloxane, methyltrimethicone, diphenylsiloxyphenyltrimethicone, phenyltrimethicone or a mixture thereof, but is not limited thereto.

The silicone oil may be comprised in an amount of about 1 to 30 wt % with respect to the total weight of the cosmetic composition. When the content of the silicone oil is out of the range, the cosmetic composition has hardness that is not suitable for being applied to the skin and thus may exhibit decreased spreadability.

For the mixture, synthetic pigment, silicone wax and mineral wax, typical materials may be purchased and used.

In addition to the aforementioned ingredients, the cosmetic composition of the present invention may further comprise a thickener, a preservative, a plasticizer, etc. within the range that does not disrupt the object and effect of the present invention.

In addition to the aforementioned active ingredients, the cosmetic composition of the present invention may comprise ingredients that are contained in a typical make-up composition, such as a dye, a nutrient component, a volatile solvent, an antioxidant, a preservative, a fragrance, etc. (hereinafter “other ingredients”), within the range that does not damage the effect of the present invention.

The cosmetic composition may have at least one formulation selected from the group consisting of a stick-type eyebrow product, a stick-type eyeliner and a stick-type eyeshadow, particularly a stick-type eyebrow product, but is not limited thereto.

Thus, the present invention can provide a stick formulation cosmetic product for eyebrow using the cosmetic composition.

Hereinafter, the present invention is described in more detail with examples. The examples are only for illustrating the present invention, and it will be obvious to a person having ordinary knowledge in the art to which the present invention pertains that the scope of the present invention is not limited thereto.

### EXAMPLE

### Preparation Example

A cosmetic composition for eye make-up having the following composition was prepared.

TABLE 1

| | Example | Example | Example | Comparative | Comparative | Comparative |
|---|---|---|---|---|---|---|
| Ingredient | 1 | 2 | 3 | Example 1 | Example 2 | Example 3 |
| Methyl | 22.5 | 22.5 | 22.5 | 22.5 | 22.5 | 22.5 |
| trimethicone | | | | | | |
| Trimethylsiloxysilicate | 22.5 | 22.5 | 22.5 | 22.5 | 22.5 | 22.5 |
| Polyethylene | 12 | 12 | 12 | 12 | 12 | 12 |
| Isohexadecane | 10.9 | 8.15 | 5.4 | 10.4 | 9.9 | 9.4 |
| Isododecane | 7.83 | 8.7875 | 11.745 | 7.83 | 7.83 | 7.83 |
| Ceresin | 4 | 4 | 4 | 4 | 4 | 4 |
| Candelilla wax | 4 | 4 | 4 | 4 | 4 | 4 |
| Hydrogenated | 3 | 3 | 3 | 3 | 3 | 3 |
| polyisobutene | | | | | | |
| Mica (CI 77019) | 2.91 | 2.91 | 2.91 | 2.91 | 2.91 | 2.91 |
| Silica | 2.88 | 2.88 | 2.88 | 2.88 | 2.88 | 2.88 |
| Rayon | 1.924 | 2.405 | 2.886 | 2.405 | 2.886 | 3.367 |
| Yellow iron oxide | 1 | 1 | 1 | 1 | 1 | 1 |
| (CI 77492) | | | | | | |
| Polyglyceryl-2 | 1 | 1 | 1 | 1 | 1 | 1 |
| triisostearate | | | | | | |
| Titanium dioxide | 1 | 1 | 1 | 1 | 1 | 1 |
| (CI 77891) | | | | | | |
| Disteardimonium | 0.9 | 1.125 | 1.35 | 0.9 | 0.9 | 0.9 |
| hectorite | | | | | | |
| Red iron oxide | 0.5 | 0.5 | 0.5 | 0.5 | 0.5 | 0.5 |
| (CI 77491) | | | | | | |
| Black iron oxide | 0.5 | 0.5 | 0.5 | 0.5 | 0.5 | 0.5 |
| (CI 77499) | | | | | | |
| Propylene carbonate | 0.27 | 3.3375 | 0.405 | 0.27 | 0.27 | 0.27 |
| Methicone | 0.12 | 0.12 | 0.12 | 0.12 | 0.12 | 0.12 |
| Dimethicone | 0.09 | 0.09 | 0.09 | 0.09 | 0.09 | 0.09 |
| Carbon black | 0.06 | 0.075 | 0.09 | 0.075 | 0.09 | 0.105 |
| (CI 77266) | | | | | | |
| Caprylyl glycol | 0.05 | 0.05 | 0.05 | 0.05 | 0.05 | 0.05 |
| Glyceryl caprylate | 0.05 | 0.05 | 0.05 | 0.05 | 0.05 | 0.05 |
| Panthenol | 0.016 | 0.02 | 0.024 | 0.02 | 0.024 | 0.028 |

A cosmetic composition having the composition as in Table 1 was prepared. The contents of the ingredients are expressed as wt %, unless specified otherwise.

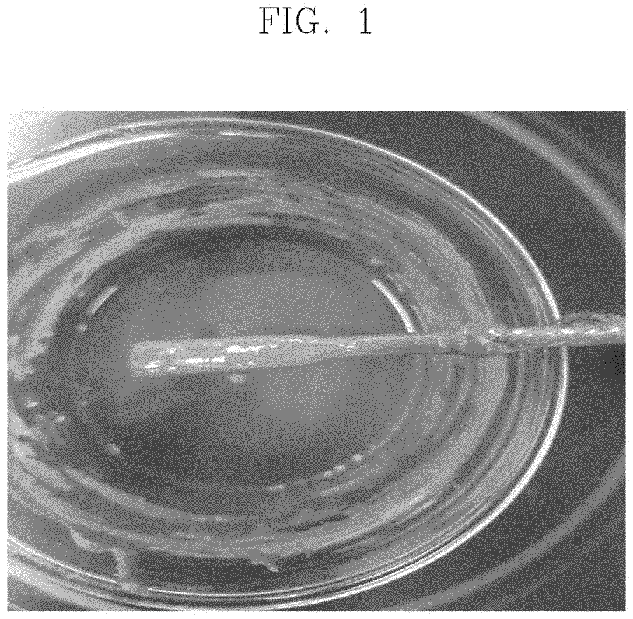

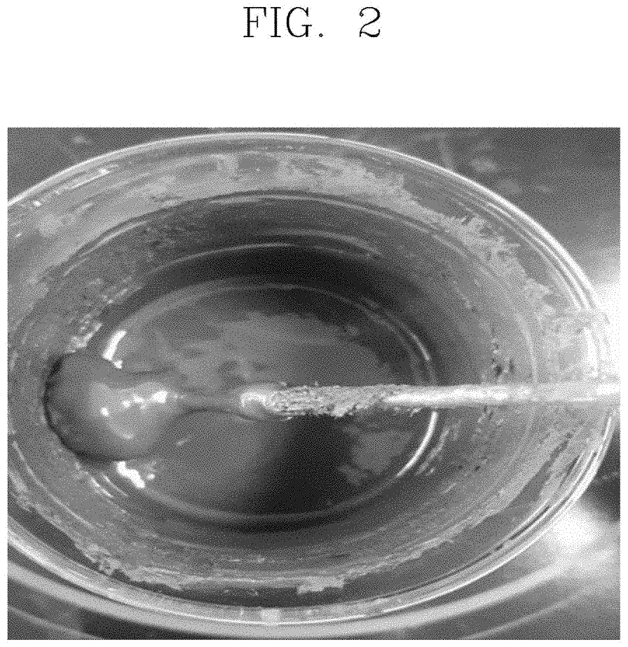

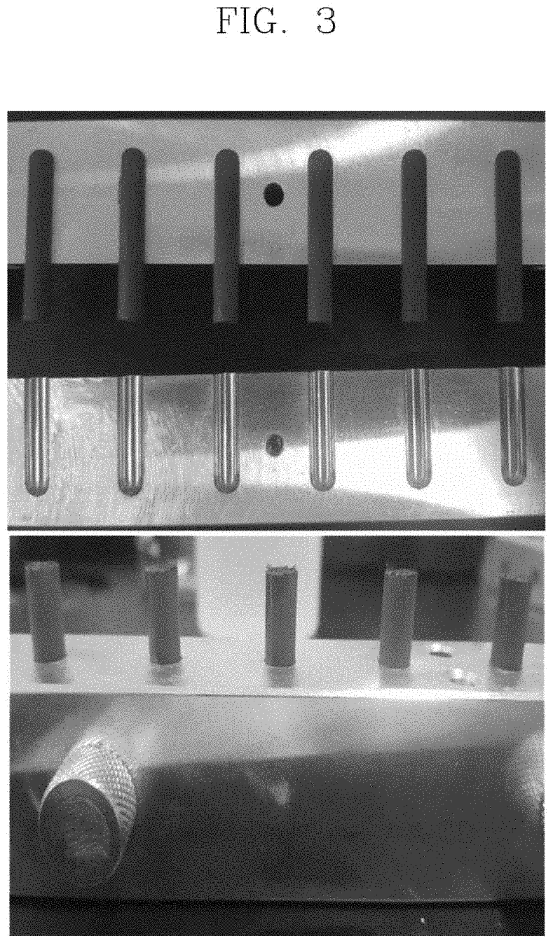

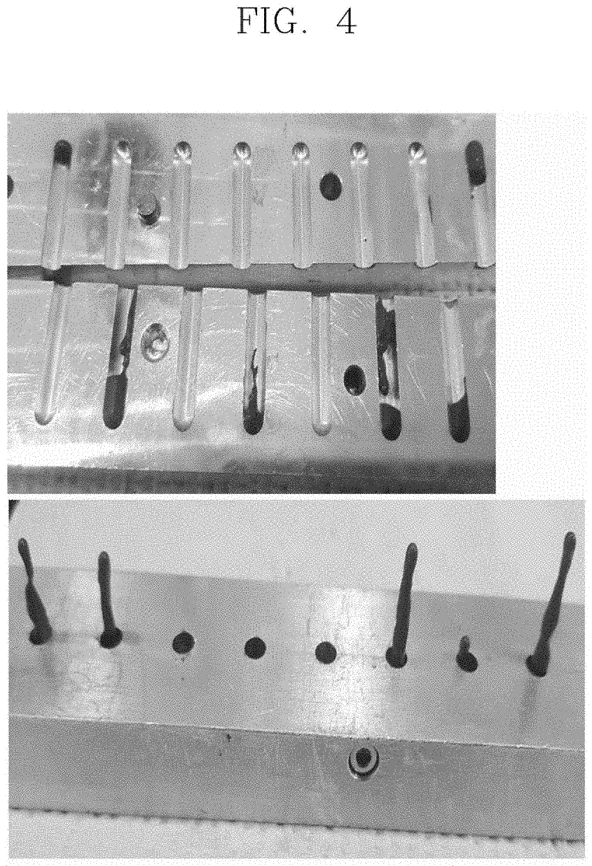

Trimethylsiloxysilicate, methyl trimethicone and disteardimonium hectorite were heated to 80° C. and dispersed at 2000 rpm for 60 minutes using a homomixer (AGI mixer). Separately, a pigment and an extender pigment were put into a food mixer and dispersed three times for 20 seconds. Other raw materials were heated to 85° C. and mixed at 1000 rpm for 20 minutes using a homomixer (AGI mixer). Then, the aforementioned mixtures were mixed together and heated to 85° C., followed by heating and mixing at 2000 rpm for 15 minutes using a homomixer (AGI mixer), to prepare a cosmetic composition. FIG. 1 and FIG. 2 show the finished compositions manufactured in Example 1 and Comparative Example 1, respectively. The prepared cosmetic compositions were molded using moldings. FIG. 3 is a photograph showing the compositions of Examples 1, 2 and 3 in upper and lower moldings, and FIG. 4 is a photograph showing the compositions of Comparative Examples 1, 2 and 3 in upper and lower moldings.

### Experimental Example 1. Appearance Evaluation According to the Ratio of Rayon and Disteardimonium Hectorite

Example 1 and Comparative Example 1 were compared to evaluate the moldability according to the ratio of rayon and disteardimonium hectorite and determine whether the ratio is essential for preparation of a stick-type cosmetic composition.

As shown in FIG. 3, in the case where the ratio of rayon and disteardimonium hectorite is about 2:1 (Example 1), the composition could be molded into a stick formulation.

By comparison, as shown in FIG. 4, in the case where the ratio of rayon and disteardimonium hectorite is about 2.5:1 (Comparative Example 1), although the hardness of the composition was not low, the composition could not be molded into a stick formulation due to agglomeration of rayon.

Thus, it was confirmed that the ratio of rayon and disteardimonium hectorite is to be 2:1 or 1:1 in order to ensure excellent moldability so as to prepare a stick formulation cosmetic composition.

According to the aforementioned result, it was confirmed that preferably, the ratio of rayon and disteardimonium hectorite is to be 2:1 in order to prepare a composition with excellent moldability that can be formulated into a specific shape such as a stick formulation.

Thus, it can be understood that the composition according to an aspect can be formulated by applying a fiber to a stick formulation, and can be dispersed and molded when the fiber and disteardimonium hectorite are comprised in a proper ratio.

### Experimental Example 2. Sensory Evaluation, Color Sustainability Evaluation and Waterproof Test on Eyebrow Composition

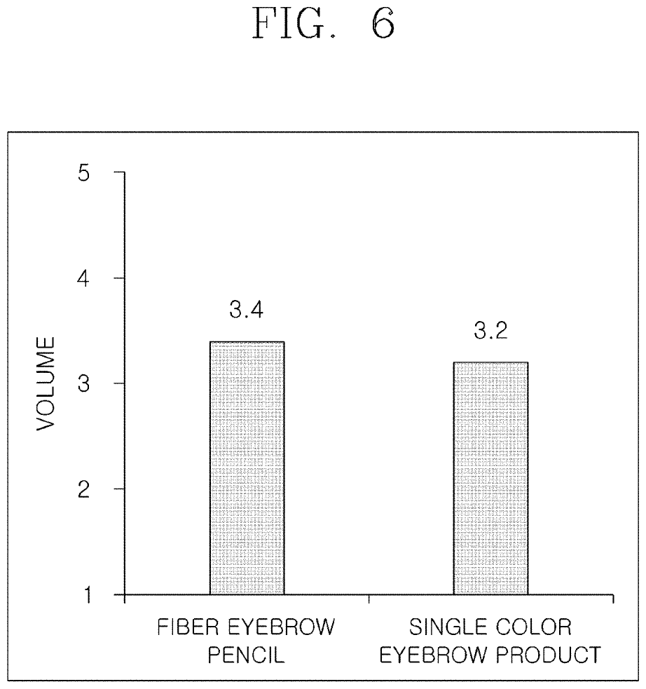

5 women in their twenties and thirties, who use eyebrow products every day or frequently, were selected to use the composition for eyebrow of Example 1 and a typical eyeliner, and photographs of their eyebrows after using the products were taken to compare the effects of the products. The test was conducted for sensory evaluation on volume, and the result is shown in FIG. 6.

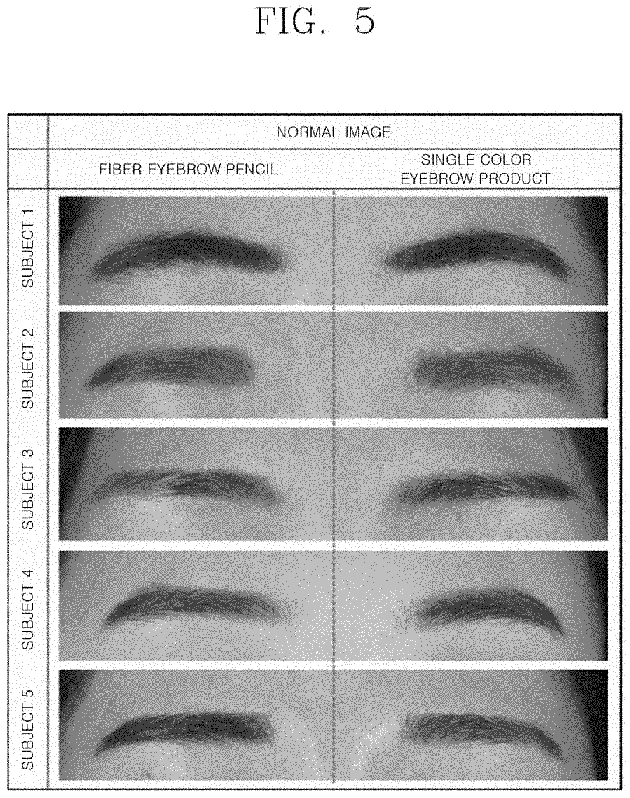

As shown in FIG. 5, it was confirmed that the composition of Example 1 provides a natural volume effect as compared with the single-color eyebrow products. FIG. 6 shows the result of sensory evaluation with the values averaged. As can be confirmed from FIG. 6, the eyebrow product manufactured with the composition of Example 1 was evaluated to have a more excellent volume effect.

### Experimental Example 3. Color Sustainability Evaluation and Waterproof Test on Eyebrow Composition

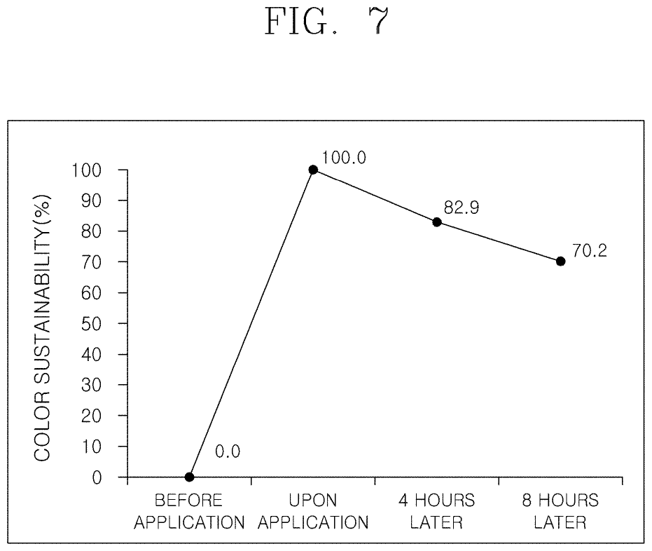

In the present experimental example, color sustainability evaluation and waterproof test were conducted. The color sustainability evaluation was conducted as follows: a photograph of the test site (eyebrows) was taken upon application of the product, the photograph was compared with photographs taken 4 hours later and 8 hours later, and L values over time were analyzed to calculate color sustainability (%) (L values indicate color brightness, and a higher L value indicates a brighter color). The result of color sustainability evaluation is shown in FIG. 7.

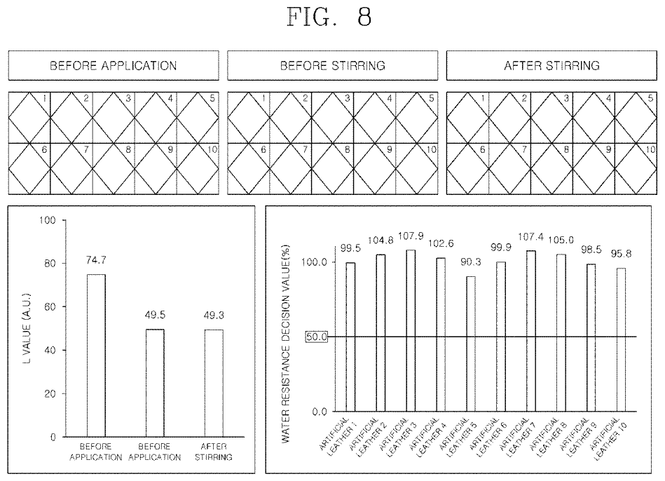

The waterproof test was conducted as follows: a photograph of 10 parts of artificial leather to which the product was applied was taken, and a photograph was taken after stirring for 20 minutes while the artificial leather was immersed in water, and L values for the two photographs were analyzed to evaluate waterproof properties (the L values of the artificial leather are analyzed, and the product is determined to have waterproof properties when all the 10 parts have L values of 50% or more). The result of waterproof test is shown in FIG. 8.

As a result, it is determined that the eyebrow product manufactured with the composition of Example 1 exhibits 82.9% color sustainability at 4 hours later and 70.2% color sustainability at 8 hours later, as shown in FIG. 7, and has waterproof properties since the L values of the 10 parts of the artificial leather remained at 50% or more as shown in FIG. 8.

The preferred examples of the present invention are described in detail as above, but the scope of the present invention is not limited thereto. It should be construed that modifications or changes of the present invention made by a person having ordinary skill in the art based on the basic concept of the present invention defined in the accompanying claims also fall within the scope of the present invention.

What is claimed is:

1. A stick formulation cosmetic composition for eye make-up comprising a fiber and a hectorite-based oil thickener.

2. The stick formulation cosmetic composition for eye make-up of claim 1, wherein the fiber and the hectorite-based oil thickener are comprised in a weight ratio of 0.1:1 to 2.5:1.

3. The stick formulation cosmetic composition for eye make-up of claim 1, wherein the composition further comprises a silicate-based film forming agent.

4. The stick formulation cosmetic composition for eye make-up of claim 1, wherein the fiber is at least one selected from the group consisting of rayon, nylon, polyester, acrylic, polyurethane and polypropylene.

5. The stick formulation cosmetic composition for eye make-up of claim 1, wherein the hectorite-based oil thickener is at least one selected from the group consisting of hectorite, disteardimonium hectorite, stearalkonium hectorite and quaternium-18 hectorite.

6. The stick formulation cosmetic composition for eye make-up of claim 1, wherein the fiber is comprised in an amount of 1 to 10 wt %.

7. The stick formulation cosmetic composition for eye make-up of claim 1, wherein the hectorite-based oil thickener is comprised in an amount of 0.1 to 5 wt %.

8. The stick formulation cosmetic composition for eye make-up of claim 1, wherein the cosmetic composition has at least one formulation selected from the group consisting of a stick-type eyebrow product, a stick-type eyeliner and a stick-type eyeshadow.

9. The stick formulation cosmetic composition for eye make-up of claim 3, wherein the silicate-based film forming agent is comprised in an amount of 1 to 30 wt %.
